(19) United States
(12) Patent Application Publication
(10) Pub. No.: US 2005/0070977 A1
(43) Pub. Date: Mar. 31, 2005

(54) Light and magnetic emitting mask

(76) Inventor: Sherry L. Molina, Charlotte, NC (US)

Correspondence Address: MORGAN & FINNEGAN, L.L.P., 3 WORLD FINANCIAL CENTER, NEW YORK, NY, 10281-2101, US

(21) Appl. No.: 10/835,618

(22) Filed: Apr. 28, 2004

Related U.S. Application Data
(60) Provisional application No. 60/465,984, filed on Apr. 28, 2003.
(60) Provisional application No. 60/480,266, filed on Jun. 20, 2003.

(51) Int. Cl. A61N 5/06

(52) U.S. Cl. 607/88; 607/100

(57) ABSTRACT

A therapeutic mask includes light emitting diodes and electromagnets imbedded in the mask. Pulsed light and pulsed magnetic energy is emitted to a user's facial region. Facial therapy is performed with the mask simultaneously or separately using magnetic therapy and light therapy. The electro-pulsed magnets are imbedded in the mask at appropriate locations based on acupressure and acupuncture therapy. Diodes in the mask are located about the center of the electro-magnetics to maximize therapeutic results. Light therapy is added to the eye area by a removable eye patch that contains light emitting diodes mounted in front of each eye. The mask can be used with a face serum-cream and an oral supplement both containing pycnogenol.

### CROSS REFERENCE TO RELATED APPLICATIONS

This application claims priority to U.S. Provisional application Ser. No. 60/465,984 filed on Apr. 28, 2003 and U.S. Provisional application Ser. No. 60/480,266 filed on Jun. 20, 2003.

### FIELD OF THE INVENTION

This invention generally relates to a medical device that delivers light and magnetic energy for therapeutic responses. More particularly, the present invention is directed to a mask that contains light emitting diodes and magnetics that deliver energy to a user's facial region for the treatment of various medical conditions.

### BACKGROUND

There are many devices for administering treatment to organic tissues for therapeutic, repair, or beauty treatment purposes. It is widely accepted that the use of light energy enhances the characteristics of the skin and provides beneficial biological and/or therapeutic results. In addition, the use of magnetic energy is also widely accepted as a therapeutic and restorative treatment to limbs, organs and other parts of the human body. However, the various therapeutic apparatus available for applying these types of energies to the skin suffer from significant structural and functional shortcomings thereby necessitating certain new and useful improvements.

For example, in U.S. Pat. No. 5,913,883 to Alexander et al., a therapeutic facial mask uniformly and totally floods light to a user's facial area using light emitting diodes. Totally flooding the skin with light can irritate the skin. This device lacks control over delivering specific energy to certain area's of treatment without irritation to the skin. The Alexander et al. device also lacks the ability to supply light therapy to the eye region. In addition, this device lacks the ability to supply magnetic energy to the skin.

In U.S. Pat. No. 6,293,900 to Bove et al. a magnetic face mask provides magneto-therapy to the eyes and/or face using static magnetic fields. Alternating magnetic fields is achieved through alternating magnetics in different patterns. Use of this device is cumbersome when therapy of various amounts of magnetic energy is desired. Different intensities and polarities of magnetic field are controlled by physically removing magnetics from the mask and replacing them with magnetics that contain the desired properties. In addition to this lack of control of the magnetic field, this device lacks the ability to supply light energy therapy.

In U.S. Pat. No. 6,520,903 to Yamashiro, an electromagnetic device generates plasma to simultaneously discharge light and magnetic energy pulses for the promotion of energy and pain relief in the human body. Again, this device lacks the control of the energy to either alternate or simultaneously direct different types of energy to the skin as desired. The device provides localized treatment and only provides treatment in one area of the body at a time. Selected treatment over a wider area is unavailable. In addition, this device lacks the ability to supply light energy to the eye region.

There remains a need for an apparatus that uses both light and electromagnetic energies in a controlled manner for therapeutic uses to the skin and eye regions. In addition, there is a need for an apparatus that offers a range of modalities and can treat multiple areas at one time without being too cumbersome or difficult to use.

### SUMMARY

The present invention provides an apparatus that uses both light and electro-magnetic energies in a controlled manner for therapeutic uses to facial skin and eye regions. The present invention is portable and easy to use. It offers a range of modalities and can treat multiple areas at one time.

A mask of the present invention includes an exterior portion and an interior portion. A plurality of light emitting diodes and a plurality of electro-magnetics are disposed about the interior portion. A control unit is coupled to the mask, either physically by a cable or wireless, for selectively controlling the light emitting diodes and the electro-magnetics.

The light emitting diodes includes white diodes, blue diodes, ruby red diodes, infra-red diodes to provide various therapeutic benefits to the skin and eyes. Different wavelengths are used for some diodes as well as different frequencies to provide the user with various options of use. The light emitting diodes are positioned about the center of the electro-magnetics for maximum therapeutic response. The light emitting diodes and electro-magnetics pulse when activated by the controller. The controller allows alternating pulses when desired by the user.

Light therapy is added to the eye area by a removable eye patch that contains light emitting diodes mounted in front of each eye. The mask can be used with a face serum-cream and an oral supplement both containing pycnogenol.

These, and other aspects of the apparatus of the present invention, are described in the following brief and detailed description of the drawings.

### BRIEF DESCRIPTION OF THE DRAWINGS

FIG. 1A is a side view of a light and magnetic emitting mask.

FIG. 1B is a front view of the mask in FIG. 1A.

FIG. 2 is an exploded perspective view of the mask in FIG. 1A.

FIG. 3 is an exploded perspective view of the mask in FIG. 1A with the addition of a gel inlay and an alternate embodiment of a strap.

FIG. 4A is a perspective interior view of the mask in FIG. 3 without the eye patch.

FIG. 4B is a perspective exterior view of the mask in FIG. 3 without the eye patch.

FIG. 5A is a partial cross-sectional view of the mask in FIG. 1A without the eye patch and strap.

FIG. 5B is an enlarged cross-sectional view of FIG. 5A.

FIG. 6A is a bottom perspective view of a controller for the mask in FIG. 1A.

FIG. 6B is a top perspective view of the controller in 6A.

FIG. 7A is a bottom perspective view of a controller for the mask in FIG. 1A.

FIG. 7B is a top perspective view of the controller in 7A.

FIG. 7C is a top view of the controller in 7A.

FIG. 8A is a top perspective and exploded view of the controller in 7A.

FIG. 8B is a bottom perspective and exploded view of the controller in 7A.

### DETAILED DESCRIPTION

This invention relates to an apparatus that includes a mask. The mask can be used for several wellness modalities. The present invention thereby provides a single structured product that can be utilized for multiple purposes. The present invention gives the user the ability to perform facial therapy with pulsed magnetic therapy and light therapy/color therapy that can be used simultaneously or separately. Typically, a user is permitted to wear the mask for about 20 minutes for each treatment and about three to four times per week.

The electro-pulsed magnets can be embedded in the mask, or protruding out from the interior of the mask. The magnets are situated at appropriate locations based on acupressure and acupuncture therapy. By activating the mask through a controller, magnetic pulses stimulate the acupuncture points which provide gateways to influence, redirect, increase, or decrease the body's vital substance, (Qi). Thus, improvements of many related conditions are possible. For example, acupuncture has been shown to stimulate the immune system. It also affects the circulation, blood pressure rhythm and stroke volume of the heart and the production of red and white cells. It also stimulates the release of a variety of hormones that helps the body respond to injury and stress. The body continually generates tiny but detectable electrical discharges. This electrical field influences the growth, maturation and functioning of some types of cells. Acupuncture points are concentrated in regions of low electrical resistance.

Acupuncture Points have certain electrical properties and stimulating these points alters chemical neurotransmitters in the body. Some theorize that individuals are affected by a so-called Magnetic Field Syndrome. Under this theory, the human body is under the influence of the earth's magnetic field and is kept in some sort of balance with it. The theory speculates that under today's living conditions the effects of this field have decreased. Consequently, we can assume that for the human body, this lack of magnetism can cause abnormalities.

By using pulsed magnetic therapy, the mask apparatus can supplement this deficiency of magnetism and abnormal conditions can improve. There is some support for this theory for a direct relationship between magnetism and improvement of abnormal conditions experienced by the human body. When NASA astronauts began getting sick on Shuttles missions from their lack of the earth's magnetic field, magnets were placed on the shuttles. After placement of the magnets, the astronauts recovered more quickly.

There are basically two types of magnetic therapy: stationary and pulse. Preferably, pulse magnetism is used. Pulse magnetism can be further divided into alternately pulsating, rotating and traveling fields. Invisible magnetic field lines permeate all the cells in the body simultaneously, down to the last molecular level, reaching parts of the body that cannot be influenced efficiently with other methods. Using pulsed magnetic therapy on acupuncture points need not be exact due to its ability to permeate the area in depth and width. There are also light emitting diodes positioned about the center of each magnet to stimulate the acupionts and further maximize the therapeutic response.

Light therapy that enters the facial skin is through the placement of light emitting diodes (LEDS) placed throughout the mask that will be further discussed. This therapy is also known as photo-modulation or photo-stimulation light therapy. The technology behind light therapy is well know by those skilled in the art and can be used for hard to heal wounds, such as diabetic skin ulcers, severe oral sores cause by chemotherapy and radiation. This process is thought to activate skin cells, thus renewing the skin. Light passes through the skin, stimulating cells directly, and the cells function more like younger cells.

The mask of the present invention also provides light therapy to the eye area. This therapy is preferably done using pulsed light therapy or photo stimulation. The light device covers the eyes and has an LED light mounted in front of each eye. Pulsed light directed in the eyes is shown to be effective against various conditions. For example, such conditions include, but are not limited to, premenstrual syndrome, headaches, seasonal affect disorder, labor, jet lag, sleep disorders and eating disorder such as bulimia nervosa. Many new light therapies have been developed. However, these devices use intense or fully flood the area of treatment. In contrast, in photo stimulation, gentle pulsed light is shone in the eyes.

The mask of the present invention covers the eyes by utilizing an eye patch that is removable. The eye patch shuts out external light. When daylight enters the eye, it reaches a gland known as the pineal gland (also known as the third eye). This daylight activates our endocrine system, which is connected to our immune systems, and can affect such systems.

Furthermore, the balance of melatonin is well known to be a crucial element in many health related conditions. Stimulating proper production of melatonin from the pineal gland is very important to good health. When the retina is stimulated with light, the light becomes a vital nutrient. At the beginning of the 20th century, approximately 70% of the Western world's population worked outdoors and benefited from exposure of natural outdoor light. Today, most people in the Western culture spend their time indoors and as a result, are missing that portion of the sun's spectrum, which is important to trigger proper melatonin secretion. It is estimated that millions of people feel the effects of malillumination (light starvation).

A lack of sunlight can lead to ill health with a variety of mental and emotional and physical symptoms. Working and living indoors, poorly illuminated environments with inappropriate artificial lighting could have serious health implications. Most artificial indoor lights lack ultraviolet light, which at the proper intensity is essential to the production of vitamin D and metabolism of calcium. Unhealthy artificial light (indoor light) lacks the requisite full color range distribution and the proper intensity to sustain health and certain functions, such as vitamin D and hormone production.

Even though light therapy is well know, light's effect on human mind and body health has been largely ignored. Both fluorescent and incandescent have lots of red, but lack green, blue and violet and indoor light generally is not bright enough, amounting to only 1/20th the intensity of outdoor lighting in the shade on a sunny day. The amount of light we receive from 16 hours indoors is dramatically less than the amount we receive outdoors. Even in locations that receive a significant amount of sun, the average person receives little sunlight in a 24 hour period. Other interferences such as, tinted glasses, tinted windows, contact lenses, do not allow in the health given properties of the entire spectrum of light. Light is a vital nutrient much like food we eat, the wrong kind can make us ill and the right kind can keep us well. Light also regulates the human body's internal biological clock. Without it our daily, monthly and annual rhythms become disrupted. Light regulates and stabilizes our physiology and emotions. Light through the eyes affects the brain and every cell in the body. Humans have biological requirements for light and it is currently unclear on how much we need of the other colors of the spectrum. Evidence points to the fact that we all can benefit from a greater supply of natural light, particularly in the winter months. Light enables us to see and plays several vital roles as it enters our eyes and skin.

Light enters the pineal gland (the body's light meter) via the retina and influences the hypothalamus. The hypothalamus is responsible for maintaining the body's status quo (the existing condition); i.e., blood pressure, body temperature, fluids and electrolyte balance and body weight. Light therapy can beneficially affect conditions related to these areas. Thus, both light therapy and magnetic therapy are desirable to deliver in a single therapeutic apparatus.

To further the benefit of these therapies, the present invention further includes a face serum-cream (for cell renewal) and an oral supplement that both contain Pycnogenol as the main ingredient. Pycnogenol, French Maritime Pine Bark extract, is a plant extract made from the bark of the European coastal pine, (pinus maritime). Pycnogenol contains approximately 40 natural ingredients, including proanthocyanidins, organic acids and other biologically active components. It has been shown to have free radical scavenging activity.

Free radicals, the normal by-products of metabolism, can accumulate and cause damage to cells and tissues. This may lead to degenerative heath and skin conditions, which are associated with the acceleration of the normal aging process. Pycnolgenol has potent antioxidant properties that protect against free radical damage. If left unchecked, free radicals, also called oxidants, can break down cells and tissue to unleash a variety of health problems. When the body is healthy, it is better able to fight off illnesses, recover from injury and put off the aging process.

Pycnogenol also has the ability to bond collagen fibers. Cells in the human body are bonded together with collagen. Pycnolgenol helps these fibers rebuild their cross-links and reverse some of the damage done due to free radicals. It also inhibits the body's enzymes that break down collagen and helps return flexibility to the skin, arteries, capillaries, joints and other tissues by restoring collagen. Pycnogenol activates vitamin C and helps it work faster, continuing to work long after Vitamin C passes out of the body. Pycnolgenol works to maintain the integrity of the blood vessels, particularly the capillaries and exerts a powerful anti-inflammatory and anti-aging effect. Pycnolgenol, improves skin elasticity and smoothness during the aging process, as well as protecting the skin against harmful effects of the sun. It has been shown to prevent LDL oxidation and to have protective action against UV radiation. Pycnolgenol has also been shown to be beneficial in cases of psoriasis. It has added value of working synergistically with many other essential nutrients.

Another element in these creams is minerals. Minerals are the regulator and activators of many cellular functions. As we age, eat poorly, experience pollution and stress, our bodies lose minerals. Without minerals we age faster, lack hydration, are prone to infections and have a sluggish metabolic energy. Minerals can cause a dramatic skin, muscular, immune and circulatory healing effects. Since minerals are small in molecular size, they are able to penetrate the skin's basil layer to replenishes de-vitalize cells. Without minerals there is no way to re-hydrate, reestablish and slow down the aging process. The skin will absorb substances that it recognizes as similar components to its own cells, like minerals.

To accomplish these benefits of light and magnetic therapies, the following example is given. This example is merely illustrative of the principles of the present invention and is not meant to limit the invention to embodiments given in this example.

TABLE 1
Description of Light Sources

| Face Therapy Light source- | Eye Therapy Light source- |
|---|---|
| LED lamps | LED lamps |
| Quantity: 128 | Quantity: 2, one per eye |
| Colors: blue, ruby red, 2 different | Color: White |
| wavelength of Infra Red (4 total colors) | |
| Intensity: adjusted by user | Intensity: adjusted by user, 8 |
| | levels of selection |
| LED drive frequencies: 73, 146, 292, | LED drive frequencies: |
| 584, 1168 Hertz, 25% Duty Cycle | 9.8, 29, 48 Hertz, Driving |
| square wave Driving method: in 4 | method: 50% Duty Cycle |
| groups, time division multiplexed | square wave, alternating |
| | left right. |

The above example demonstrates that the facial area is treated with at least four different types of light treatment. Typically, the red light treatment is directed to destroying acne and other related skin maladies. The blue light treatment is directed to destroying harmful bacteria. The infra red light treatment provides benefits already discussed as does the white light treatment for the eyes. Pulsed light energy is used to reduce irritation to the skin and eyes during application of the treatment.

The following is an illustrative example of the specifications of LEDs that can be used in the present invention. Again, this example is given as a mere illustration, and is not intended to limit the invention to this particular embodiment.

TABLE 2
Examples of LEDs for Facial Treatment

| | Infrared LED | | Ruby Red LED | |
|---|---|---|---|---|
| Infrared LED | option 2 | Ruby Red LED | option 2 | Blue LED |
| Device: Stanley | Device: Unity | Device: Fema | Device: LITEON | Device: Fema |
| DNP1105W | Opto Technology | LRF2SC-R | C930KFKT | LRF2BC-R |
| | MSE-1542H2 | | | |
| Wavelength: | Wavelength: | Wavelength: | Wavelength: | Wavelength: |
| 880 nm | 850 nm | 639 nm | 611 nm | 467 nm |
| Light source | Light source | Light source | Light source | Light source |
| distance to skin: | distance to skin: | distance to skin: | distance to skin: | distance to skin: |
| 0.6 cm | 0.6 cm | 0.6 cm | 0.6 cm | 0.6 cm |
| Treatment time: = | Treatment time: = | Treatment time: = | Treatment time: = | Treatment time: = |
| 375 seconds | 375 seconds | 375 seconds | 375 seconds | 375 seconds |
| Light output | Light output | Light output | Light output | Light output |
| energy density = | energy density = | energy density = | energy density = | energy density = |
| 4.166 Joule/cm² | 8.3325 Joule/cm² | 1.562 Joule/cm² | 3.645 Joule/cm² | 1.177 Joule/cm² |

As indicated in the above example, various wavelengths are possible to use with the light treatment of the present invention. In the above example the wavelengths ranged from about 467 nm to about 880 nm. The above example used a test time of 375 seconds and a distance to skin of 0.6 cm. As stated before, the LEDs used in the present invention are not so limited to such a distance from skin. The calculated light out put energy density for the above example was up to about 8.5 Joule/cm². It was further noted that the use of (32) Ruby Red LED and (32) Blue LEDs yielded an average light output energy density of 2.767 Joule/cm² and the use of (64) Infrared. (32) Red, (32) Blue LEDs yielded an average light output energy density of 5.372 Joule/cm² as determined by the below equations:
Ee_{avg}((2×Ee_{IR})+Eep+Ee_{BLUE})/4=((2×4.166)+1.562+1.177)/4)=2.767 Joule/cm² (Equation 1)
Ee_{avg}((2×EeI_{R})+Ee_{RED}+Ee_{BLUE})/4=((2×8.3325)+3.645+1.177)/4)=5372 Joule/cm² (Equation 2)

In these equations, Ee_{avg} is the average light output energy density, Ee_{IR}, Eep, Ee_{BLUE}, Ee_{RED} are light out put density from the above Table 2 for LEDs of infra-red, ruby red, blue, and ruby red option 2, respectively.

The following is an example of the electromagnets. Again, these examples are merely given for illustrative purposes. The present invention is not limited to the embodiments illustrated.

TABLE 3
Description of Electro-magnets
Face Therapy Electro-Magnet per Light source-LED lamps
Quantity: 1
Construction: air core
Intensity: adjusted by user
Drive method: 50% Duty Cycle 16 Hz square wave

As illustrated in the above example, 1 magnet is disposed per each LED disposed in the interior of the mask. Preferably, the LED is about in the center of the electromagnet to maximize the therapeutic response of the light and magnetic therapy.

Adverting to the drawings, FIGS. 1A & 1B illustrates a side view and a front view, respectively, of mask 2. The mask includes an exterior portion 5 and an interior portion 7. The mask can be made of many layers or made of a single unitary layer, depending on the implementation. As shown in FIG. 1A, mask 2 has an exterior layer 4 that includes exterior portion 5.

An eye patch 6 is provided to deliver light therapy to the eyes if the user so desires. The eye path is removably attached to the exterior portion 5 by an eye therapy connection port 10.

Breathing holes 12, allow the user to wear the mask comfortably for an extended period of time. In addition the breathing holes permit the use of aroma therapy with the light and magnetic therapy. Voice through hole 14 allows the user to communicate while wearing the mask. Users of the mask can essentially continue their daily routine while the mask is delivering therapy.

A control unit connection port 16 allows the mask 2 to be connected to a controller. The connection can be by a cable (not shown fully extended) or by a wireless connection know to those skilled in the art. This flexibility in connection to a controller allows the user to be mobile while receiving therapy from the mask.

Straps 20 are shown to fix the mask on a user's facial region. However, any type of fixing apparatus can be used with the invention. These types of fixing apparatus include but are not limited to hoods, elastic bands, string, rope, wire frames, a unitary molded helmet, and the like. In the illustrative example of FIG. 1A, straps 20 are attached to the mask by a metal loop 24 and contain a strap ring 22. The strap is adjustable by the strap loop and/or ring. Again any means of fixing the mask about the user can be used with the present invention.

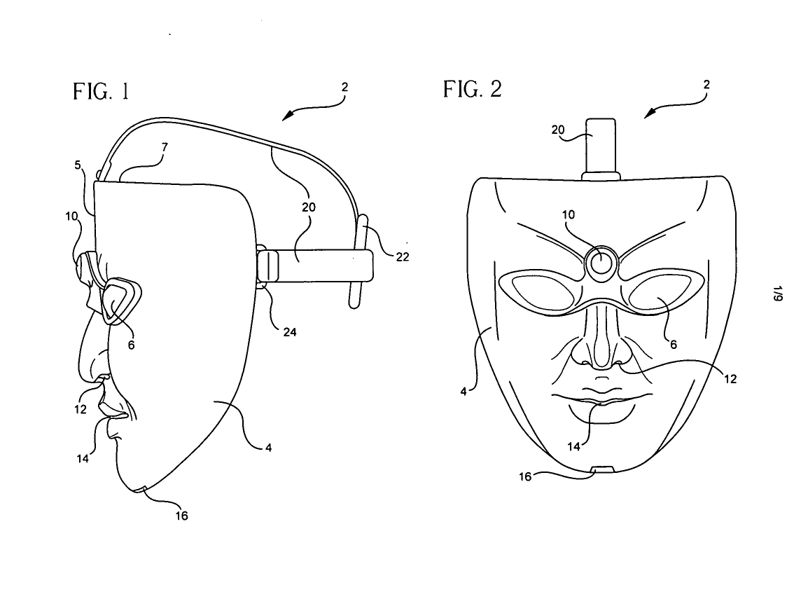

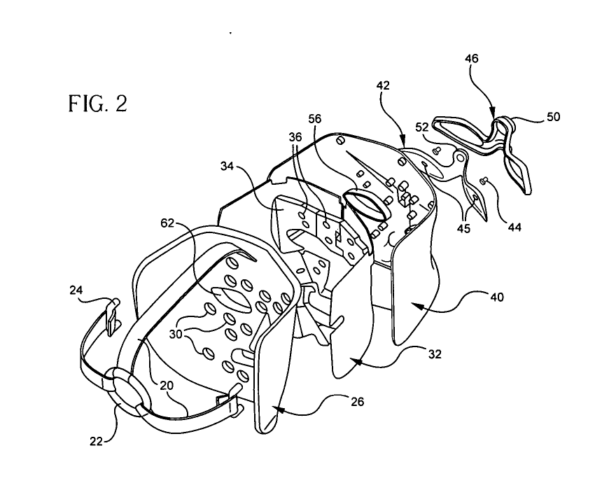

FIG. 2 illustrates an exploded view of the mask. Inner mask 26 faces the user and is attached to strap 20. The inner mask defines through holes 30 for the magnetics and LEDs to pass through to the user. The distance from the LEDs and magnetics to the user's skin is typically a predetermined distance. However, it is within the purview of this invention that the distance can be adjusted if so desired by the user by simply adjusting the straps to pull the mask closer or farther away. Inner mask 26 also includes through holes 62 for the user's eyes and eye light therapy.

Attached to the inner mask is a flexible printed circuit board 32. Flexible Printed circuit board 32 includes a printed circuit board (PCB) 34. The PCB contains the LEDs and magnetics used in the therapy. Additionally, depending on the implementation the PCB may be configured to control the LEDs and the magnetics such that the controller is integral with the mask. For example, the controller port 16 may be a control console for the control of the LEDs and magnetics disposed on the PCB.

Typically attached to the flexible printed circuit board 32 is an exterior mask 40 to provide protection. The exterior mask 40 includes through holes 56 for eye light therapy/Eye light therapy is delivered by LEDs 44. Preferably, LEDs 44 are white pulse LEDs as previously described to prevent irritation to the eyes. LEDs 44 are attached to inner eye patch 42 through holes 45. An exterior eye patch 46 may cover the assembly and provide protection. Attachment of the eye patch to the mask is done by an eye connection port 50 or eye connection port 52. It should be understood that the eye patch can be a single unitary and integral component that does not require interior and exterior layers as previously described.

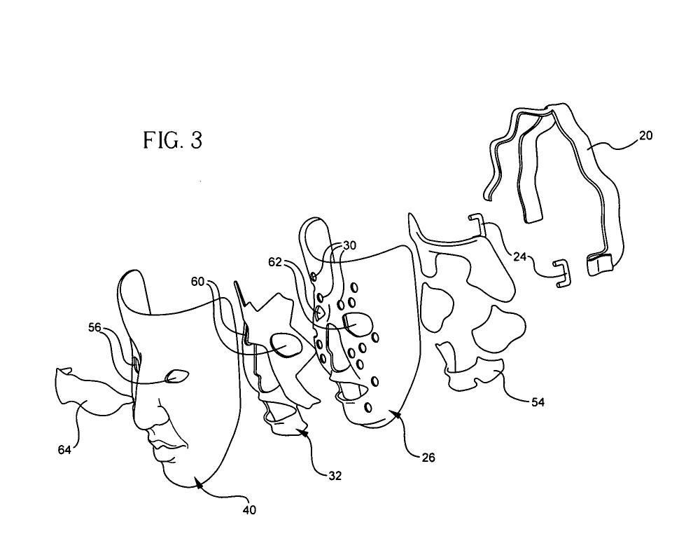

FIG. 3 illustrates another exploded view of the mask from its front. Illustrated in FIG. 3 is a gel inlay that is typically attached to the back of inner mask 26 or a similar structure. The gel inlay provides comfort for the user and may contain trans-dermal medication to further the therapy of the skin. Also illustrated in FIG. 3 is the removal of the strap ring 22. Adjustments to the strap can be done through loops 24, or by any other suitable means. Also illustrated in FIG. 3 is a single piece eye patch 64 that contains LEDs 44 (not shown in FIG. 3). The flexible printed circuit board 32 defines eye through holes 60 for facilitating eye light therapy from the eye patch LED's.

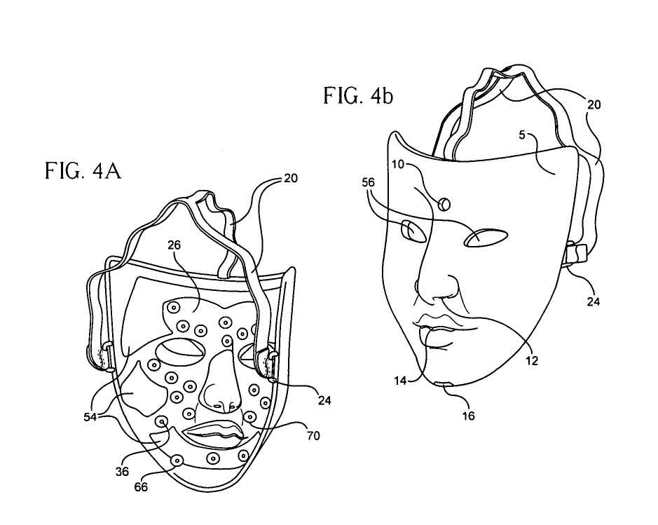

FIG. 4A illustrates the back of the mask. Shown is the gel inlay 54 interior portion 7 of the mask and LED/magnetic 36. Again, preferably LED 66 of the facial mask is positioned about the center of magnet 70 to maximize the therapeutic response. FIG. 4B illustrates the front portion of the mask in 4A, and demonstrates the mask without the eye patch attachment that connects to port 10 on exterior mask 4.

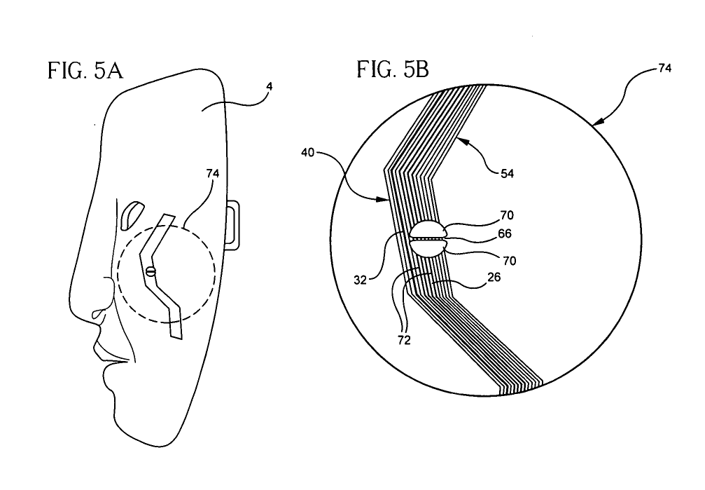

As shown in FIG. 5A, cross section 74 of the assembled mask can consist of several layers. Again, depending on the implementation, the mask may be comprised of an integral layer containing components from each previously described layer. In FIG. 5b, LED 66 is disposed about the center of magnet 70. Typically one LED is utilized in the mask per one magnet, however, many variations can be used in the present invention, and the mask of the present invention is not limited to the illustrations shown. Through holes 72 allows space for the LEDs and magnetics to pass through each successive layer without hindrance.

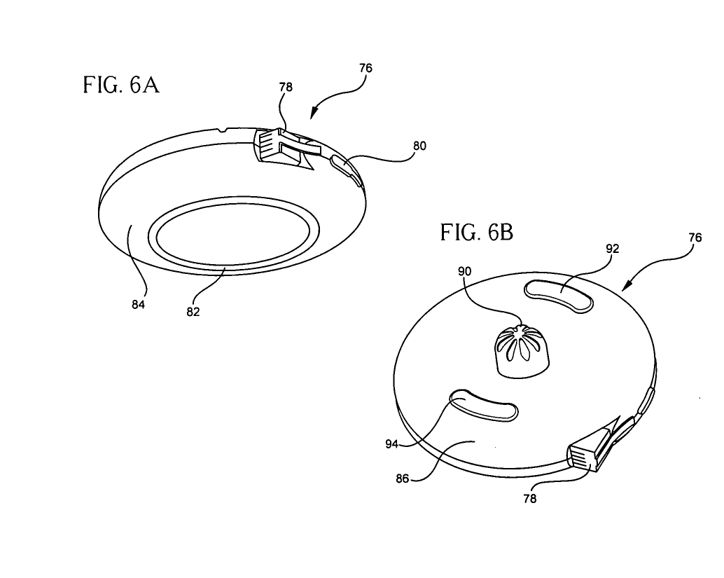

Averting to the control unit, shown in FIG. 6A is one embodiment of control unit 76. In this illustration a cable (not shown) is retracted within the housing of the control unit. A release button 80 can release the cable from the control unit housing. The communication plug 78 is attached to the cable. Alternatively, communication plug 78 can be a wireless communication device known to those skilled in the art to communicate with the mask. As previously stated the control unit may be integrally made part of the mask and place on any portion of the mask. A power source 82 can be part of the control unit 76 or an external power source can be utilized. For example, DC batteries such (4) AA battery could last for 2-3 hours. Alternatively, an externally powered (DC or AC jack) can be used or a LAN jack (R345) can be used for the mask's electrical connections.

Controller 76 has a bottom 84 and a top 86. The control panel illustrated in FIG. 6B is on top 86. A start/intensity magnetic pulse adjustment knob 90 is disposed about the center of controller 76. This knob adjusts the intensity of the magnetic pulse and starts the magnetic therapy, whether it is magnetic therapy alone or both magnetic and light therapy. A light therapy start button 94 allows the user to activate the facial LEDs, either alone or in conjunction with the magnetics. An eye light start button 92 allows the eye light therapy to be activated. There is an automatic shut off control feature in the controller that allows the user with added safety in using the mask.

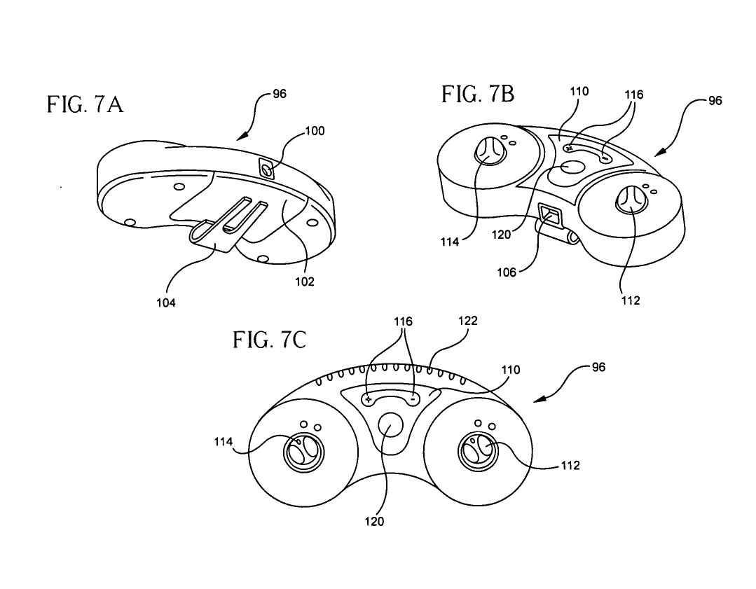

FIGS. 7A-7C illustrate a controller 96 embodiment. Controller 96 has a DC or AC optional connector for external power. A battery cover 102 protects a battery compartment for an internal power source. The cover may also include a clip 104 so that the controller can be attached to the user's belt or clothing further making the mask easy to use and allowing the user to be mobile while receiving therapy from the mask. The mask communication port can attach to a cable (not shown) to the port 16 of the mask. Alternatively there can be a wireless connection between port 106 and port 16 as previously discussed.

Controller 96 has a general control pad 110. Intensity control 116 allows control of the intensity of the therapy chosen. A mode control button 120 allows control over what type of therapy the user wants delivered. For example, the user can have the following exemplary options for therapy. Therapy options include, but are not limited to, 1. light therapy to the face region only, 2. magnetic therapy to the face region only, 3. light therapy to the face region and magnetic therapy to the face region together, and alternating, 4. light therapy to the eye region only, and 5. magnetic therapy to the face region and eye light therapy together (simultaneously).

Individual control to the magnetic light therapies can be given by magnetic therapy control knob 112, and light therapy control knob 114. These control knobs control the intensity and duration of each respective type of therapy. The eye light control is controlled by control 116 and mode control 120. In addition, it is within the scope of the present invention that the controller can choose through user input what frequency of light or magnetic energy the user will receive, what intensity and duration. Air vents 122 can be provide to assist in cooling the controller. Optionally or in addition to, a heat sink can be place within the controller.

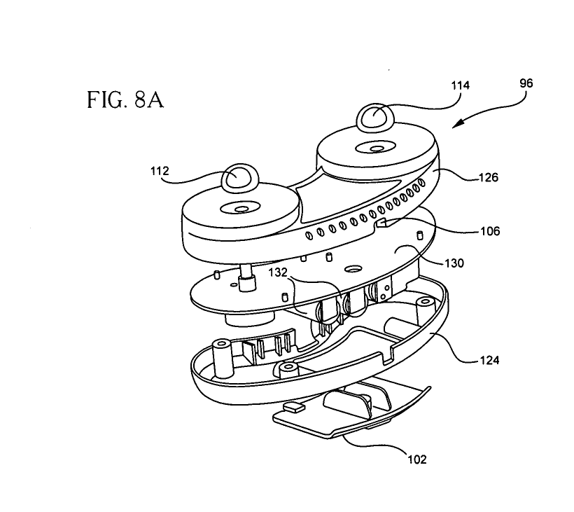

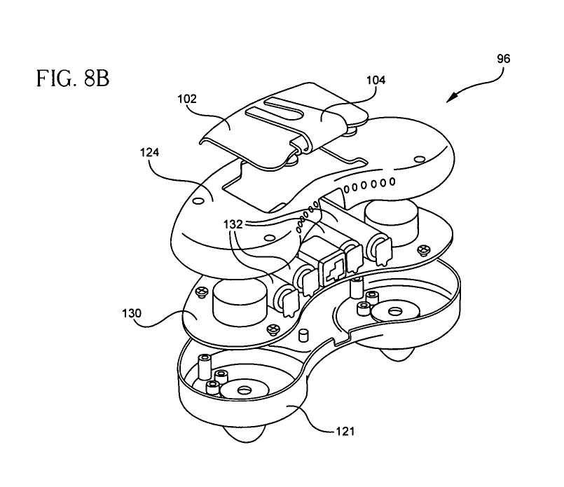

FIGS. 8A and 8B illustrate an exploded view of controller 96. Shown is a bottom cover 124, a top cover 126, and a printed circuit board 130. Power can be external or internal as shown by batteries 132. The printed circuit board 130 can be configured to control the mask's delivery of light and magnetic therapies so that the user has selective control over all aspects of these therapies and over what areas the user wants exposed to the specific therapy. For example, a user may have acne on the chin. The user could program the controller to apply light energy only to the chin area. Alternatively, the user may want high energy in one area, and low energy in another. In addition, the user may want just eye light therapy. The user may desire facial magnetic therapy and eye light therapy. The user may desire facial light therapy, eye light therapy, and facial magnetic therapy. All of these variations are possible with use of the controller. Several more and different variations are available and are all within the scope of the present invention.

The following is an exemplary description of the operation of the mask. Again, this description is meant as an example, and does not limit the invention to such operation sequences. The present invention can utilize many different operating sequences to deliver the desired therapy to the user of the mask.

The description of operation includes turning either the face/eye or magnetic therapy rotary switch from off position to the on position. The mask through the controller unit will enter into a very low power, standby mode after about 25 minutes, in magnetic/face therapy mode, and after about 16 minutes in the eye therapy mode.

After sleep mode, the unit restarts by repositioning either switch. The eye therapy module wakes up from sleep mode by pressing the mode button. It's possible to change therapy modes during sessions while not affecting the remaining time, however only turning both knobs to power off position, for at least 20 seconds, will reset the unit.

To de-energize the unit, both rotary switches should be in off position. There are four indicator lights showing the activity of each therapy options. If only “face therapy” is selected, 32 LEDs will be active at the time for 11.76 seconds in a 1 to 4 time division multiplex mode. The duty cycle therefore is 25% (128 LEDs). There are four groups of LEDs, 32 of them in each group (128 total LEDs). Every group is active for 11.76 seconds then the program rotates the four groups by 255 times, for a total of 25 minutes. The LEDs are also driven at five different frequencies (73, 146, 292, 584, 1168 Hz), each active for 5 minutes.

If only “magnetic pulse” therapy is selected the program drives the coil for 25 minutes, at 16 Hz, 50% duty cycle, with the pre-selected polarity. If both the magnetic and the face light therapy are selected, the program will alternate between the two modes. Each mode is active for about 5.88 seconds at the time, for 255 times (total 25 min). The coil and LEDs are driven the same way as described before.

The “eye light” therapy can be selected alone or only together with “magnetic pulse” therapy. The “eye light” with “magnetic pulse” therapy will run simultaneously without any interaction. The eye light therapy is enabled in the last position of “face/eye” rotary switch. This time the three pushbuttons are active. The “mode” button will cycle between the three different frequencies (9.8, 29, 49 Hz) and the “brightness up”/“brightness down” buttons select the eight of light output levels.

It should be understood that the above description is only representative of illustrative examples of embodiments. For the reader's convenience, the above description has focused on a representative sample of all possible embodiments, a sample that teaches the principles of the invention. Other embodiments may result from a different combination of portions of different embodiments. The description has not attempted to exhaustively enumerate all possible variations.

Furthermore, since numerous modifications and variations will readily occur to those skilled in the art, it is not desired that the present invention be limited to the exact construction and operation illustrated. Accordingly, all suitable modifications and equivalents that may be resorted to are intended to fall within the scope of the claims.

1. An apparatus for producing a therapeutic response, comprising:
a mask having an exterior portion and an interior portion,
a plurality of light emitting diodes and a plurality of electro-magnetics disposed about the interior portion, and
a control unit coupled to the mask for selectively controlling the light emitting diodes and the electro-magnetics.

2. The apparatus of claim 1, wherein the light emitting diodes further includes blue diodes, ruby red diodes, infra-red diodes or any combination thereof.

3. The apparatus of claim 2, wherein the infrared diodes have at least two different wavelengths.

4. The apparatus of claim 3, wherein all the light emitting diodes are driven at five different frequencies.

5. The apparatus of claim 1, wherein the light emitting diodes are positioned about the center of the electro-magnetics for maximum therapeutic response.

6. The apparatus of claim 1, wherein the light emitting diodes and electro-magnetics pulse when activated by the controller.

7. The apparatus of claim 6, wherein the light emitting diodes and electro-magnetics alternately pulse when activated by the controller.

8. An apparatus for producing a therapeutic response, comprising:
a mask having an exterior portion and an interior portion,
a plurality of light emitting diodes positioned about centers of a plurality of electro-magnetics, the diodes and the electro-magnetics disposed about the interior portion,
an eye patch removably attached to the exterior of the mask, the eye patch further including at least two light emitting diodes that provides light to eyes of a user, and
a control unit coupled to the mask for selectively controlling the light emitting diodes, the electro-magnetics, and the light emitting diodes of the eye patch.

9. The apparatus of claim 8, wherein the light emitting diodes of the interior portion of the mask includes blue diodes, ruby red diodes and infra-red diodes of at least two different wavelengths, and the light emitting diodes of the eye patch are white diodes.

10. The apparatus of claim 8, wherein the control unit is configured to allow activation of only the light emitting diodes in the interior portion of the mask.

11. The apparatus of claim 8, wherein the control unit is configured to allow only activation of only the electro-magnetics in the interior portion of the mask.

12. The apparatus of claim 8, wherein the control unit is configured to allow activation of only the light emitting diodes and the electro-magnetics in the interior portion of the mask together.

13. The apparatus of claim 12, wherein the light emitting diodes and electro-magnetics pulse and are alternately activated.

14. The apparatus of claim 8, wherein the control unit is configured to allow activation of only the light emitting diodes of the eye patch.

15. The apparatus of claim 8, wherein the control unit is configured to allow activation of the light emitting diodes of the eye patch and the electro-magnetics in the interior portion of the mask.

16. The apparatus of claim 15, wherein the light emitting diodes of the eye patch and the electro-magnetics are simultaneously activated together.

17. The apparatus of claim 8, wherein the control unit is configured to allow activation of the light emitting diodes of the eye patch and the interior portion of the mask together.

18. The apparatus of claim 8, wherein the control unit is configured to allow activation of the light emitting diodes of the eye patch, the interior portion of the mask, and the electro-magnetics in the interior portion of the mask together.

19. The apparatus of claim 8, wherein the controller is configured to allow preset energy delivery from the light emitting diodes of the interior portion of the mask and the eye patch and the electro-magnetics.

20. The apparatus of claim 19 wherein the controller further includes an automatic shut off that terminates energy delivery after a predetermined time period.

21. An apparatus for producing a therapeutic response, comprising:
an external mask portion having a mask having an exterior portion and an interior portion, the exterior portion having an eye therapy connection port and a controller connection port,
a flexible circuit board attached to the interior portion of the external mask and in communication with the controller connection port, the flexible circuit board having a plurality of light emitting diodes positioned about centers of a plurality of electro-magnetics, the diodes and the electro-magnetics disposed about the flexible circuit board,
an internal mask attached to the flexible circuit board to provide support to the circuit board, the diodes, and the electro-magnetics,
a gel inlay attached to the internal mask and positioned opposite the flexible circuit board for providing facial comfort to a user,
an eye patch removably attached to the eye therapy connection port, the eye patch further including at least two light emitting diodes that provides light to eyes of the user, and
a control unit coupled to the controller connection port of the external mask for selectively controlling the light emitting diodes, the electro-magnetics, and the light emitting diodes of the eye patch.

22. The apparatus of claim 21, wherein the control unit is coupled to the controller connection port of the external mask by wireless connection.

23. The apparatus of claim 21, wherein the control unit is coupled to the controller connection port of the external mask by a cable.

24. The apparatus of claim 23, wherein the cable is retractable within the control unit.

25. The apparatus of claim 21, wherein the external mask further defines a nasal aperture for breathing and a mouth aperture for speaking by the user.

26. The apparatus of claim 21, wherein the external mask further includes a strap for adjustably securing the apparatus over a facial area of the user.

27. The apparatus of claim 21, wherein the controller further includes a printed circuit board that is configured to adjust the intensity of the light emitting diodes of the eye patch, the diodes of the interior portion of the external mask, and the electro-magnetics.

28. The apparatus of claim 21, wherein the light emitting diodes of the interior portion are blue diodes, ruby red diodes, and two different wavelength infra-red diodes.

29. The apparatus of claim 21, wherein the diodes of the eye patch are white diodes.

30. The apparatus of claim 21, wherein the average light output energy density of the light emitting diodes of the interior portion is up to about 8.5 Joule/cm².

31. A kit containing an apparatus for producing a therapeutic response, comprising:
a face serum-cream,
a mask having an exterior portion and an interior portion,
a plurality of light emitting diodes and a plurality of electro-magnetics disposed about the interior portion, and
a control unit coupled to the mask for selectively controlling the light emitting diodes and the electro-magnetics.

32. The kit of claim 31, wherein the face serum-cream is pycnogenol.

33. The kit of claim 31, further including an oral supplement containing at least pycnogenol.

34. An apparatus for producing a therapeutic response, comprising:
a mask having an exterior portion and an interior portion,
a plurality of light emitting diodes disposed about the interior portion that releases pulsed light energy, the light emitting diodes consisting of blue diodes, ruby red diodes, and infra-red diodes of at least two different wavelengths and any combination thereof, and
a control unit coupled to the mask for selectively controlling the light emitting diodes and configured to drive the diodes at least at five different frequencies.

35. The apparatus of claim 34, further including a plurality of pulsed electro-magnetics disposed around the diodes.

36. An apparatus for producing a therapeutic response, comprising:
a mask having an exterior portion and an interior portion,
a plurality of pulsed electro-magnetics disposed about the interior portion, the electro-magnetics arranged at specific acupuncture and acupressure points to facilitate a therapeutic response, and
a control unit coupled to the mask for selectively controlling the electro-magnetics and configured to drive the electro-magnetics for a predetermined time period.

37. The apparatus of claim 36, further including pulsed light emitting diodes disposed about the center of the electro-magnetics for facilitating light therapy.

38. The apparatus of claim 36, further including a gel in lay attached to the interior portion, the gel inlay containing a trans-dermal medication for delivery of medication to a treatment site.

39. The apparatus of claim 36, wherein the mask further defines a breathing hole to allow the use of aroma therapy.
